(19) United States
(12) Patent Application Publication
(10) Pub. No.: US 2008/0318331 A1
(43) Pub. Date: Dec. 25, 2008

(54) Anthraquinone Derivatives as Markers For Liquids

(75) Inventors: Rüdiger Sens, Ludwigshafen (DE); Thomas Gessner, Heidelberg (DE); Sophia Ebert, Mannheim (DE); Christos Vamvakaris, Mannheim (DE); Wolfgang Ahlers, Lambsheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/159,330

(22) PCT Filed: Dec. 12, 2007
(86) PCT No.: PCT/EP2007/063813

§ 371 Date: Jun. 26, 2008

(30) Foreign Application Priority Data
Dec. 20, 2006 (EP) 06126725.8

(51) Int. Cl. G01N 33/00; C07D 209/56

(52) U.S. Cl. 436/96; 548/426

(57) ABSTRACT

The use of compounds of the general formula (I)

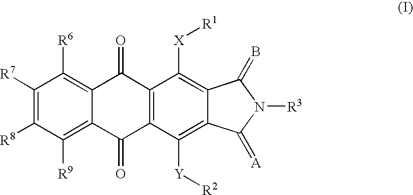

as markers for liquids, methods for detecting markers in liquids, methods for identifying liquids and selected compounds of the general formula (I).

The present invention relates to the use of compounds of the general formula (I)

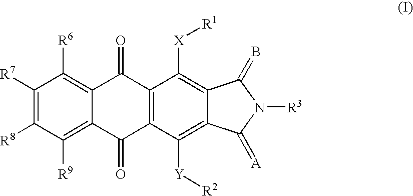

as markers for liquids, where the symbols are each defined as follows:
X, Y are each independently, identically or differently, O, NR⁴, heterocycles,
A, B are each independently, identically or differently, O, NR⁵,
M are alkali metals,
R¹, R² are each independently, identically or differently, H, C₁-C₂₀-alkyl, C₂-C₂₀-alkenyl, C₂-C₂₀-alkynyl, C₃-C₁₅-cycloalkyl, aryl, heterocycles,
R³ is C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, heterocycles,
R⁴ is H, C₁-C₂₀-alkyl, C₂-C₂₀-alkenyl, C₂-C₂₀-alkynyl, C₃-C₁₅-cycloalkyl, aryl, heterocycles,
R⁵ is H, C₁-C₂₀-alkyl, C₂-C₂₀-alkenyl, C₂-C₂₀-alkynyl, C₃-C₁₅-cycloalkyl, aryl, heterocycles,
R⁶, R⁷, R⁸, R⁹ are each independently, identically or differently, H, C₁-C₂₀-alkyl, C₁-C₂₀-alkylcarbonyl, C₁-C₂₀-alkoxy, C₁-C₂₀-alkoxycarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, aryloxy, aryloxycarbonyl, heterocycles, NR¹R², halogen, CN, NO₂,
   where the substituents R¹, R², R³, R⁴, R⁵, R⁶, R⁷, R⁸ or R⁹ may each be interrupted at any position by one or more heteroatoms, where the number of these heteroatoms is not more than 10, preferably not more than 8, more preferably not more than 5 and especially not more than 3, and/or may be substituted in each case at any position, but not more than five times, preferably not more than four times and more preferably not more than three times, by NR¹R², CONR¹R², COOM, COOR¹, SO₃M, SO₃R¹, CN, NO₂, C₁-C₂₀-alkyl, C₁-C₂₀-alkoxy, aryl, aryloxy, heterocycles, heteroatoms or halogen, where these may likewise be substituted not more than twice, preferably not more than once, by the groups mentioned.

The invention further relates to liquids which comprise a compound of the general formula (I) as a marker. The invention further relates to methods for detecting markers in liquids and for identifying liquids which comprise at least one compound of the general formula (I). The invention further relates to various novel compounds of the general formula (I).

Further embodiments of the present invention can be taken from the claims, the description and the examples. It is self-evident that the aforementioned features, and the features which are still to be mentioned below, of the inventive subject matter are usable not only in the combination stated specifically in each case but also in other combinations without leaving the scope of the invention. Preferred and very preferred embodiments of the present invention are in particular also those in which all features of the inventive subject matter have the preferred and very preferred meanings.

Particular anthraquinone derivatives and their use as markers for mineral oil products are known.

U.S. Pat. No. 3,164,449 describes anthraquinone derivatives and their use as markers for mineral oils. The anthraquinone derivatives described in this document do not comprise compounds of the general formula (I).

EP 1 001 003 A1 describes a method for the invisible marking of mineral oil products with the aid of dyes. These dyes may, inter alia, also be particular 1,4-substituted anthraquinone derivatives which do not, however, comprise compounds of the general formula (I).

Documents EP 1 323 811 A2, EP 1 422 284 A2, EP 1 426 434 A2, EP 1 479 749 A1 and EP 1 486 554 A1 disclose methods for marking mineral oils by adding various anthraquinone derivatives. They are, for example, 1,4,5,8-tetrasubstituted anthraquinones or anthraquinone dimers with absorption maxima in the range from 710 to 850 nm, or tetra- to octasubstituted anthraquinones with absorption maxima in the range from 690 to 1000 nm. Mixtures of different anthraquinone derivatives are likewise described. The anthraquinone derivatives disclosed in these documents do not comprise compounds of the general formula (I).

WO 2005/063942 A1 discloses fuel and lubricant concentrates which comprise at least one anthraquinone derivative as a marker. Compounds of the general formula (I) are not described as markers.

Particular compounds of the general formula (I), especially specific anthraquinonedicarboximides, and processes for their preparation are known.

DE 939 044 and DE 945 112 describe processes for preparing 1,4-diamino-2,3-anthraquinonedicarboximides substituted on the imide nitrogen atom and their use in the dyeing of polyethylene terephthalate fibers. A use of these compounds as markers for liquids is not disclosed.

DE 1 176 777 describes the preparation of particular anthraquinonedicarboximides proceeding from 1-amino-4-nitroanthraquinone-2-carboxylic acid. A use of these compounds as markers for liquids is not disclosed.

In practice, it is found that many of the known markers, especially in mineral oils, with the additives typically present therein, or in additive concentrates, often do not have the desired long-term stability. The action of said additives changes, for example, the spectral properties (e.g. absorbance) of the markers. Frequently, precise detection of the markers and reliable identification of the liquids, especially at low marker concentrations, is therefore possible only to a limited degree after prolonged periods.

It was therefore an object of the invention to provide further anthraquinone derivatives and related compounds which feature not only good solubility but also good long-term stability and storage stability in the liquids to be marked, especially mineral oils or additive concentrates.

Detection of the markers frequently takes place with the aid of spectroscopic methods by detecting the absorption or the fluorescence. The higher the fluorescence quantum yield of the markers, the more sensitively detection itself can be effected at low concentration of the marker. It was therefore a further object of the invention to find markers which have an increased quantum yield compared to the known markers, especially the anthraquinones used for the marking of mineral oils.

The markers can be detected at different temperatures. It is therefore necessary to find markers which can be detected as independently of temperature as possible or with reproducible known temperature dependence.

It has been found that the above-described compounds of the general formula (I), for example anthraquinonedicarboximides and related compounds, have both a good solubility and a very good long-term stability, especially compared to customary fuel additives. Moreover, especially anthraquinonedicarboximides which are among the above-described compounds of the general formula (I) are notable for an elevated fluorescence quantum yield compared to the anthraquinone derivatives used as mineral oil markers in the prior art. The temperature dependence found for the fluorescence is frequently very low for the compounds of the general formula (I).

In the context of this invention, expressions of the form Cₐ-C_{b} denote chemical compounds or substituents having a particular number of carbon atoms. The number of carbon atoms can be selected from the entire range from a to b, including a and b; a is at least 1 and b is always greater than a. A further specification of the chemical compounds or of the substituents is effected by expressions of the form Cₐ-C_{b}—V. In this case, V represents a chemical compound class or substituent class, for example alkyl compounds or alkyl substituents.

Halogen represents fluorine, chlorine, bromine or iodine, preferably fluorine, chlorine or bromine, more preferably fluorine or chlorine.

Alkali metals are Li, Na or K. In particular, the alkali metals (M) in the chemical group —SO₃M or —COOM may occur as monovalent positively charged ions.

Specifically, the collective terms specified for the different substituents R¹, R², R³, R⁴, R⁵, R⁶, R⁷, R⁸, R⁹, R¹⁰, R¹¹, X, Y, A and B are each defined as follows:

C₁-C₂₀-Alkyl: straight-chain or branched hydrocarbon radicals having up to 20 carbon atoms, for example C₁-C₁₀-alkyl or C₁₁-C₂₀-alkyl, preferably C₁-C₁₀-alkyl, for example C₁-C₃-alkyl, such as methyl, ethyl, propyl, isopropyl, or C₄-C₆-alkyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methyl-pentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-tri-methylpropyl, 1-ethyl-1-methylpropyl, 1-ethyl-2-methylpropyl, or C₇-C₁₀-alkyl such as heptyl, octyl, 2-ethylhexyl, 2,4,4-trimethylpentyl, 1,1,3,3-tetramethylbutyl, nonyl or decyl, and isomers thereof.

C₁-C₂₀-Alkylcarbonyl: a straight-chain or branched alkyl group having from 1 to 20 carbon atoms (as specified above) which is attached via a carbonyl group (—CO—), preferably C₁-C₁₀-alkylcarbonyl, for example formyl, acetyl, n- or isopropionyl, n-, iso-, sec- or tert-butanoyl, n-iso-, sec- or tert-pentanoyl, n- or isononanoyl, n-dodecanoyl.

C₁-C₂₀-Alkoxy means a straight-chain or branched alkyl group having from 1 to 20 carbon atoms (as specified above) which is attached via an oxygen atom (—O—), for example C₁-C₁₀-alkoxy or C₁₁-C₂₀-alkoxy, preferably C₁-C₁₀-alkyloxy, especially preferably C₁-C₃-alkoxy, for example methoxy, ethoxy, propoxy.

C₁-C₂₀-Alkoxycarbonyl: is an alkoxy group having from 1 to 20 carbon atoms (as specified above) which is attached via a carbonyl group (—CO—), preferably C₁-C₁₀-alkyloxycarbonyl.

C₂-C₂₀-Alkenyl: unsaturated, straight-chain or branched hydrocarbon radicals having from 2 to 20 carbon atoms and a double bond in any position, for example C₂-C₁₀-alkenyl or C₁₁-C₂₀-alkenyl, preferably C₂-C₁₀-alkenyl such as C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, or C₅-C₆-alkenyl, such as 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl or 1-ethyl-2-methyl-2-propenyl, and also C₇-C₁₀-alkenyl, such as the isomers of heptenyl, octenyl, nonenyl or decenyl.

C₂-C₂₀-Alkenylcarbonyl: unsaturated, straight-chain or branched hydrocarbon radicals having from 2 to 20 carbon atoms and a double bond in any position (as specified above), which are attached via a carbonyl group (—CO—), preferably C₂-C₁₀-alkylcarbonyl, for example ethenoyl, propenoyl, butenoyl, pentenoyl, nonenoyl and isomers thereof.

C₂-C₂₀-Alkynyl: straight-chain or branched hydrocarbon groups having from 2 to 20 carbon atoms and a triple bond in any position, for example C₂-C₁₀-alkynyl or C₁₁-C₂₀-alkynyl, preferably C₂-C₁₀-alkynyl such as C₂-C₄-alkynyl, such as ethynyl, 1-propynyl, 2-propynyl, 1-butynyl, 2-butynyl, 3-butynyl, 1-methyl-2-propynyl, or C₅-C₇-alkynyl, such as 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl or 1-ethyl-1-methyl-2-propynyl, and C₇-C₁₀-alkynyl such as the isomers of heptynyl, octynyl, nonynyl, decynyl.

C₂-C₂₀-Alkynylcarbonyl: unsaturated, straight-chain or branched hydrocarbon radicals having from 2 to 20 carbon atoms and a triple bond in any position (as specified above), which are attached via a carbonyl group (—CO—), preferably C₂-C₁₀-alkynylcarbonyl, for example propynoyl, butynoyl, pentynoyl, nonynoyl, decynoyl and isomers thereof.

C₃-C₁₅-Cycloalkyl: monocyclic saturated hydrocarbon groups having from 3 up to 15 carbon ring members, preferably C₃-C₈-cycloalkyl such as cyclopropyl, cyclobutyl, cyclopentyl, cyclohexyl, cycloheptyl or cyclooctyl, and a saturated or unsaturated cyclic system, for example norbornyl or norbenzyl.

C₃-C₁₅-Cycloalkylcarbonyl: monocyclic saturated hydrocarbon groups having from 3 to 15 carbon ring members (as specified above), which are attached via a carbonyl group (—CO—), preferably C₃-C₈-cycloalkylcarbonyl.

Aryl: a mono- to tricyclic aromatic ring system comprising from 6 to 14 carbon ring members, for example phenyl, naphthyl or anthracenyl, preferably a mono- to bicyclic, more preferably a monocyclic, aromatic ring system.

Arylcarbonyl: preferably a mono- to tricyclic aromatic ring system (as specified above) which is attached via a carbonyl group (—CO—), for example benzoyl, preferably a mono- to bicyclic, more preferably a monocyclic, aromatic ring system.

Aryloxy: is a mono- to tricyclic aromatic ring system (as specified above) which is attached via an oxygen atom (—O—), preferably a mono- to bicyclic, more preferably a monocyclic, aromatic ring system.

Aryloxycarbonyl: is a mono- to tricyclic aryloxy group (as specified above) which is attached via a carbonyl group (—CO—), preferably a mono- to bicyclic, more preferably a monocyclic, aryloxycarbonyl.

Heterocycles: five- to twelve-membered, preferably five- to nine-membered, more preferably five- to six-membered, ring systems having oxygen, nitrogen and/or sulfur atoms and optionally a plurality of rings, such as furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl. The heterocycles may be attached chemically to the compounds of the general formula (I) in any manner, for example via a bond to a carbon atom of the heterocycle or a bond to one of the heteroatoms. Moreover, especially five- or six-membered saturated nitrogen-containing ring systems which are attached via a ring nitrogen atom and which may also comprise one or two further nitrogen atoms or a further oxygen or sulfur atom.

COOR¹: represents carboxylic acids (R¹═H) or carboxylic esters (where, for example, R¹═C₁-C₂₀-alkyl or aryl).

COOM: represents salts of carboxylic acids (for example monovalent alkali metal salts).

SO₃R¹: represents sulfonic acids (R¹═H) or sulfonic esters (where, for example, R¹═C₁-C₂₀-alkyl or aryl).

SO₃M: represents salts of sulfonic acids (for example monovalent alkali metal salts).

CONR¹R²: represents optionally substituted carboxamides. For example, in this case, R¹ and R² are identically or differently C₁-C₂₀-alkyl or aryl.

Heteroatoms are phosphorus, oxygen, nitrogen or sulfur, preferably oxygen, nitrogen or sulfur.

The symbols in formula (I) are preferably each defined as follows:
X, Y are each independently, identically or differently, O, NR⁴,
A, B are each independently, identically or differently, NH, O,
R¹, R² are identically or differently H, C₁-C₂₀-alkyl, aryl,
R³ is C₁-C₂₀-alkyl, C₃-C₁₅-cycloalkyl, aryl, heterocycles,
R⁴ is H, C₁-C₂₀-alkyl, aryl, heterocycles,
R⁶, R⁷, R⁸, R⁹ are each independently, identically or differently, H, C₁-C₂₀-alkyl, C₁-C₂₀-alkoxy, C₃-C₁₅-cycloalkyl, aryl, aryloxy, NR¹R², halogen, CN, NO₂.

Very preferably, the symbols in formula (I) are each defined as follows:
X, Y are both identically NR⁴,
A, B are each independently, identically or differently, NH, O,
R¹, R² are each H,
R³ is C₁-C₂₀-alkyl, C₃-C₁₅-cycloalkyl, aryl,
R⁴ is H,
R⁶, R⁷, R⁸, R⁹ are each independently, identically or differently, H, C₁-C₂₀-alkyl, C₁-C₂₀-alkoxy, aryl, aryloxy, NR¹R², F, Cl, Br, CN, NO₂.

Likewise preferably, the symbols in formula (I) are each defined as follows:
X, Y are both identically NR⁴,
A, B are each O,
R¹, R² are each H,
R³ is C₁-C₂₀-alkyl, C₃-C₁₅-cycloalkyl, aryl,
R⁴ is H,
R⁶, R⁷, R⁸, R⁹ are each independently, identically or differently, H, C₁-C₂₀-alkyl, C₁-C₂₀-alkoxy, aryl, aryloxy, NR¹R², F, Cl, Br, CN, NO₂.

Preferred and very preferred is the use of compounds of the general formula (I) in which all symbols have, respectively, the preferred and very preferred definitions.

The markers used in the process according to the invention may be either individual compounds of the general formula (I) or mixtures of compounds of the general formula (I).

The compounds of the general formula (I) can be prepared by methods familiar to those skilled in the art, as described, for example, in M. C. Marschalk, Bull. Soc. Chim. 1937, 184-193, DE 1 769 470, DE 1 176 777, DE 939 044 and DE 945 112.

Some of the compounds of the general formula (I) are known and some of them are novel.

The invention therefore also provides, inter alia, compounds of the general formula (I) in which the symbols in formula (I) are each defined as follows:
X, Y are each NH,
A, B are each O,
R¹, R² are each H,
R³ is C₁₃-C₂₀-alkyl,
R⁶, R⁷, R⁸, R⁹ are each H.

The invention especially preferably provides the compound where R³═C₁₋₃-alkyl: 1,4-diamino-N-tridecyl-2,3-anthraquinonedicarboximide. The tridecyl substituent in this compound may of course also be an isomer mixture of different tridecyls.

The invention further preferably provides the compounds 1,4-diamino-N-(2,6-diisopropylphenyl)-2,3-anthraquinonedicarboximide or 1,4-diamino-N-(4-dodecylphenyl)-2,3-anthraquinonedicarboximide, and also mixtures of these compounds. The dodecyl substituent in the compound 1,4-diamino-N-(4-dodecylphenyl)-2,3-anthraquinonedicarboximide may of course also be an isomer mixture of different dodecyls.

Suitable liquids which can be marked by means of the compounds of the general formula (I) in accordance with the process according to the invention are in particular water or organic liquids, for example alcohols such as methanol, ethanol, propanol, isopropanol, butanol, isobutanol, sec-butanol, pentanol, isopentanol, neopentanol or hexanol, glycols such as 1,2-ethylene glycol, 1,2- or 1,3-propylene glycol, 1,2-, 2,3- or 1,4-butylene glycol, di- or triethylene glycol or di- or tripropylene glycol, ethers such as methyl tert-butyl ether, 1,2-ethylene glycol monomethyl or -dimethyl ether, 1,2-ethylene glycol monoethyl or diethyl ether, 3-methoxypropanol, 3-isopropoxypropanol, tetrahydrofuran or dioxane, ketones, such as acetone, methyl ethyl ketone or diacetone alcohol, esters such as methyl acetate, ethyl acetate, propyl acetate or butyl acetate, aliphatic or aromatic hydrocarbons such as pentane, hexane, heptane, octane, isooctane, petroleum ether, toluene, xylene, ethylbenzene, tetralin, decalin, dimethylnaphthalene, petroleum spirit, brake fluids or oils such as mineral oils which, in accordance with the invention, comprise gasoline, kerosene, diesel oil and heating oil, natural oils such as olive oil, soybean oil or sunflower oil, or natural or synthetic motor, hydraulic or transmission oils, for example vehicle motor oil or sewing machine oil.

Particularly advantageously, the compounds of the general formula (I) are used in accordance with the process according to the invention for the marking of oils, especially mineral oils.

The invention further provides liquids, preferably oils, especially mineral oils, which comprise at least one compound of the general formula (I) as a marker.

The compounds of the general formula (I) to be used as markers are added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb.

To mark the liquids, the compounds are added generally in the form of solutions (stock solutions). Especially in the case of mineral oils, suitable solvents for preparing these stock solutions are preferably aromatic hydrocarbons such as toluene, xylene or relatively high-boiling aromatic mixtures.

In order to avoid an excessively high viscosity of such inventive stock solutions (and hence poor metering and handling), a total concentration of the markers of from 0.5 to 50% by weight, preferably from 0.5 to 40% by weight, more preferably from 0.5 to 30% by weight, based on the total weight of these stock solutions, is generally selected.

The compounds of the general formula (I) may, if appropriate, also be used in mixtures with other markers/dyes. In that case, the total amount of the markers in the liquids is typically within the above-described range.

The invention also provides a process for marking liquids, preferably oils, especially mineral oils, wherein a compound of the general formula (I) is added to the liquid.

The invention also provides a method for detecting markers in liquids which comprise at least one compound of the general formula (I).

The compounds of the general formula (I) in the liquids are detected by common methods known to those skilled in the art. Since the compounds of the general formula (I) generally have a high absorption capacity and/or exhibit high fluorescence quantum yield, one example of a possibility in the given case is spectroscopic detection. In this context, reference is made explicitly to the disclosure of the documents WO 94/02570 (page 14 lines 10-46 and FIG. 1), WO 99/55805 (page 22 line 7-page 34 line 46) and WO 99/56125 (page 22 line 22-page 46 line 15).

The compounds of the general formula (I) generally have their absorption maximum in the range from 500 to 900 nm and/or fluoresce in the range from 500 to 1000 nm and can thus be detected easily with suitable instruments.

The detection can be carried out in a manner known per se, for example by measuring the absorption spectrum of the liquids to be analyzed.

However, it is also advantageously possible to excite the fluorescence of the compounds of the general formula (I) present in the liquids, advantageously with a semiconductor laser or a semiconductor diode. It is particularly advantageous to employ a semiconductor laser or a semiconductor diode with a wavelength in the spectral range from λₘₐₓ−100 nm to λₘₐₓ+20 nm. λₘₐₓ means the wavelength of the longest-wavelength absorption maximum of the marker. The wavelength of maximum emission is generally in the range from 500 to 900 nm.

The fluorescence light thus generated is advantageously detected with a semiconductor detector, especially with a silicon photodiode or a germanium photodiode.

The detection succeeds particularly advantageously when an interference filter and/or an edge filter (with a short-wavelength transmission edge in the range from λₘₐₓ to λₘₐₓ+80 nm) and/or a polarizer is disposed upstream of the detector. By means of the abovementioned compounds, it is possible in a very simple manner to detect marked liquids even when the compounds of the general formula (I) are present only in a concentration of about 1 ppm (detection by absorption) or about 100 ppb (detection by fluorescence).

A preferred method for detecting markers in liquids which comprise at least one compound of the general formula (I) in an amount which is sufficient to excite detectable fluorescence on irradiation with radiation of a suitable wavelength is performed by:
a) irradiating the liquid with electromagnetic radiation of a wavelength of from 500 to 900 nm and
b) detecting the excited fluorescence radiation with a device for detecting radiation in the range from 500 to 1000 nm.

A further preferred process for detecting markers in liquids which comprise at least one compound of the general formula (I) in an amount which is sufficient to exhibit detectable absorption on irradiation with radiation of a suitable wavelength is performed by:
a) irradiating the liquid with electromagnetic radiation of a wavelength of from 500 to 900 nm and
b) detecting the absorption of the radiation a) with a device for detecting radiation in the range from 500 to 900 nm.

The invention also provides a method for identifying liquids, preferably oils, especially mineral oils, which comprise a compound of the general formula (I) in an amount which is sufficient to excite detectable fluorescence on irradiation with a suitable wavelength, wherein
a) the liquid is irradiated with electromagnetic radiation of a wavelength of from 500 to 900 nm and
b) the absorption of the electromagnetic radiation a) is detected with a device for detecting radiation and
c) the excited fluorescence radiation is detected with a device for detecting radiation in the range from 500 to 900 nm and
d) the liquid is identified with the aid of the absorption b) and/or fluorescence
c) and
e) the concentration of the compound of the general formula (I) in the liquid is determined with the aid of fluorescence radiation c).

In a preferred embodiment of the method according to the invention for identification, the measurement data from steps b) and e) of the process are combined in order to perform the identification. The identification may comprise, as a further step, comparison with known spectroscopic data. For example, the known spectroscopic data are electronically stored spectra which may be deposited, for example, in databases.

If mixtures of compounds of the general formula (I) are used as markers, the wavelength positions of the absorption maxima λₘₐₓ preferably differ by a spectroscopically measurable magnitude. The positions of the absorption maxima preferably differ in each case by the magnitude of at least 40 nm. λₘₐₓ means the wavelength of the longest-wavelength absorption maximum of the marker.

In general, the absorption bands of the compounds of the general formula (I) may either overlap or be present separately from one another. Controlled detection and the combined detection of a plurality of spectroscopic properties, for example in absorption or fluorescence, allows so-called “fingerprint systems” to be built up in the case of the inventive use of mixtures of compounds of the general formula (I).

It is preferably also possible to utilize different quantitative ratios in a mixture of the compounds of the general formula (I) to obtain different markers. It is preferably possible with two compounds V¹ and V² of the general formula (I) which are present in n different quantitative ratios to obtain n markers for the inventive use (n is an integer greater than 1). For example, in the case that n=2, it is possible with the quantitative ratios of V¹:V²=1:2 and V¹:V²=2:1 to obtain two markers. The particular quantitative ratios can be determined by the person skilled in the art with the aid of simple routine experiments for the particular use.

In a preferred embodiment of the process according to the invention, as well as compounds of the general formula (I), further (at least one) marker substances (MA) other than the compounds of the general formula (I) are used as markers. In such a mixture, preference is given to using those markers (MA) whose absorption maximum λₘₐₓ is at a wavelength which differs by at least 40 nm from the position of the absorption maxima of the compounds of the general formula (I) which occur in the mixture. In such a mixture, preference is given to using markers (MA) whose λₘₐₓ is at a wavelength which is greater (longer in wavelength) than that of the compounds of the general formula (I). However, it is also possible in such a mixture to use markers (MA) whose λₘₐₓ is at a wavelength which is smaller (shorter in wavelength) than that of the compounds of the general formula (I).

In general, the absorption bands of the markers (MA) and of the compounds of the general formula (I) may either overlap or be present separately from one another. Controlled detection and the combined detection of a plurality of spectroscopic properties, for example in absorption or fluorescence, allows so-called “fingerprint systems” to be built up in the case of the inventive use of mixtures of the markers (MA) and of the compounds of the general formula (I).

It is preferably also possible to utilize different quantitative ratios in a mixture of the markers (MA) and of the compounds of the general formula (I) to obtain different markers. It is preferably possible with two compounds MA¹ of the markers (MA) and V² of the general formula (I) which are present in n different quantitative ratios to obtain n markers for the inventive use (n is an integer greater than 1). For example, in the case that n=2, it is possible to obtain two markers with the quantitative ratios MA¹:V²=1:2 and MA¹:V²=2:1.

The quantitative ratio of markers (MA) to compounds of the general formula (I) is generally dependent upon the particular use and the detection sensitivity of the marker (MA). The particular quantitative ratios can be determined for the particular use by the person skilled in the art with reference to simple routine experiments.

Possible markers (MA) include anthraquinones other than the compounds of the general formula (I), phthalocyanines (metal-free and metal-containing) or naphthalocyanines. Preference is given to using anthraquinones other than the compounds of the general formula (I) or phthalocyanines, and more preferably phthalocyanines.

The compounds of the general formula (I) may also be used as a component in additive concentrates (also referred to hereinafter, following the relevant terminology, as “packages”), which, as well as a carrier oil and a mixture of different fuel additives, generally also comprise dyes and, for the invisible fiscal or manufacturer-specific marking, additionally markers. These packages enable various mineral oil distributors to be supplied from a “pool” of unadditized mineral oil, and only with the aid of their individual packages are the company-specific additization, color and marking imparted to the mineral oil, for example during the filling into appropriate transport vessels.

The components present in such inventive packages are then in particular:
a) at least one compound of the general formula (I),
b) at least one carrier oil,
c) at least one additive selected from the group consisting of
   i. detergents,
   ii. dispersants and
   iii. valve seat wear-inhibiting additives,
d) and also, if appropriate, further additives and assistants.

The carrier oils used are typically viscous, high-boiling and in particular thermally stable liquids. They cover the hot metal surfaces, for example the intake valves, with a thin liquid film and thus prevent or delay the formation and deposition of decomposition products on the metal surfaces.

Carrier oils useful as component b) of the fuel and lubricant additive concentrates are, for example, mineral carrier oils (base oils), especially those of the Solvent Neutral (SN) 500 to 2000 viscosity class, synthetic carrier oils based on olefin polymers having M_{N}=from 400 to 1800, in particular based on polybutene or polyisobutene (hydrogenated or nonhydrogenated), on poly-alpha-olefins or poly(internal olefins) and also synthetic carrier oils based on alkoxylated long-chain alcohols or phenols. Adducts, to be used as carrier oils, of ethylene oxide, propylene oxide and/or butylene oxide to polybutyl alcohols or polyisobutene alcohols are described, for instance, in EP 277 345 A1; further polyalkene alcohol polyalkoxylates to be used are described in WO 00/50543 A1. Further carrier oils to be used also include polyalkene alcohol polyether amines, as detailed in WO 00/61708.

It is of course also possible to use mixtures of different carrier oils, as long as they are compatible with one another and with the remaining components of the packages.

Carburetors and intake systems of internal combustion engines, but also injection systems for fuel metering, are being contaminated to an increasing degree by impurities which are caused, for example, by dust particles from the air and uncombusted hydro-carbons from the combustion chamber.

To reduce or prevent these contaminations, additives (“detergents”) are added to the fuel to keep valves and carburetors or injection systems clean. Such detergents are generally used in combination with one or more carrier oils. The carrier oils exert an additional “wash function”, support and often promote the detergents in their action of cleaning and keeping clean, and can thus contribute to the reduction in the amount of detergents required.

It should also be mentioned here that many of the substances typically used as carrier oils display additional action as detergents and/or dispersants, which is why the proportion of the latter can be reduced in such a case. Such carrier oils having detergent/dispersant action are detailed, for instance, in the last-mentioned WO document. It is also often impossible to clearly delimit the mode of action of detergents, dispersants and valve seat wear-inhibiting additives, which is why these compounds are listed in summary under component c). Customary detergents which find use in the packages are listed, for example, in WO 00/50543 A1 and WO 00/61708 A1 and comprise:

polyisobuteneamines which are obtainable according to EP-A 244 616 by hydro-formylation of highly reactive polyisobutene and subsequent reductive amination with ammonia, monoamines or polyamines, such as dimethyleneaminopropylamine, ethylenediamine, diethylenetriamine, triethylenetetramine or tetraethylenepentamine,
poly(iso)buteneamines which are obtainable by chlorination of polybutenes or polyisobutenes having double bonds predominantly in the β- and γ-position and subsequent amination with ammonia, monoamines or the abovementioned polyamines,
poly(iso)buteneamines which are obtainable by oxidation of double bonds in poly(iso)butenes with air or ozone to give carbonyl or carboxyl compounds and subsequent amination under reducing (hydrogenating) conditions,
polyisobuteneamines which are obtainable according to DE-A 196 20 262 from polyisobutene epoxides by reaction with amines and subsequent dehydration and reduction of the amino alcohols,
polyisobuteneamines which optionally comprise hydroxyl groups and are obtainable according to WO-A 97/03946 by reaction of polyisobutenes having an average degree of polymerization P of from 5 to 100 with nitrogen oxides or mixtures of nitrogen oxides and oxygen and subsequent hydrogenation of these reaction products,
polyisobuteneamines which comprise hydroxyl groups and are obtainable according to EP-A 476 485 by reaction of polyisobutene epoxides with ammonia, monoamines or the abovementioned polyamines,
polyetheramines which are obtainable by reaction of C₂- to C₃₀-alkanols, C₆- to C₃₀-alkanediols, mono- or di-C₂- to C₃₀-alkylamines, C₁- to C₃₀-alkylcyclohexanols or C₁- to C₃₀-alkylphenols with from 1 to 30 mol of ethylene oxide and/or propylene oxide and/or butylene oxide per hydroxyl or amino group and subsequent reductive amination with ammonia, monoamines or the abovementioned polyamines, and also
“polyisobutene Mannich bases” which are obtainable according to EP-A 831 141 by reaction of polyisobutene-substituted phenols with aldehydes and monoamines or the abovementioned polyamines.

Further detergents and/or valve seat wear-inhibiting additives to be used are listed, for example, in WO 00/47698 A1 and comprise compounds which have at least one hydrophobic hydrocarbon radical having a number-average molecular weight (M_{N}) of from 85 to 20 000 and at least one polar moiety, and which are selected from:
(i) mono- or polyamino groups having up to 6 nitrogen atoms, of which at least one nitrogen atom has basic properties;
(ii) nitro groups, optionally in combination with hydroxyl groups;
(iii) hydroxyl groups in combination with mono- or polyamino groups, in which at least one nitrogen atom has basic properties;
(iv) carboxyl groups or their alkali metal or alkaline earth metal salts;
(v) sulfonic acid groups or their alkali metal or alkaline earth metal salts;
(vi) polyoxy-C₂- to —C₄-alkylene moieties which are terminated by hydroxyl groups, mono- or polyamino groups, in which at least one nitrogen atom has basic properties, or by carbamate groups;
(vii) carboxylic ester groups;
(viii) moieties derived from succinic anhydride and having hydroxyl and/or amino and/or amido and/or imido groups; and
(ix) moieties obtained by Mannich reaction of phenolic hydroxyl groups with aldehydes and mono- or polyamines.

Additives comprising mono- or polyamino groups (i) are preferably polyalkenemono- or polyalkenepolyamines based on polypropene or on highly reactive (i.e. having pre-dominantly terminal double bonds, usually in the β- and γ-positions) or conventional (i.e. having predominantly internal double bonds) polybutene or polyisobutene having M_{N}=from 300 to 5000. Such additives based on highly reactive polyisobutene, which can be prepared from the polyisobutene (which may comprise up to 20% by weight of n-butene units) by hydroformylation and reductive amination with ammonia, monoamines or polyamines, such as dimethylaminopropylamine, ethylenediamine, diethylenetriamine, triethylenetetramine or tetraethylenepentamine, are disclosed in particular in EP 244 616 A2. When polybutene or polyisobutene having predominantly internal double bonds (usually in the β- and γ-positions) is used as starting material in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be the same as those used above for the reductive amination of the hydroformylated highly reactive polyisobutene. Corresponding additives based on polypropene are described in particular in WO 94/24231 A1.

Further preferred additives comprising monoamino groups (i) are the hydrogenation products of the reaction products of polyisobutenes having an average degree of polymerization P of from 5 to 100 with nitrogen oxides or mixtures of nitrogen oxides and oxygen, as described in particular in WO 97/03946 A1.

Further preferred additives comprising monoamino groups (i) are the compounds obtainable from polyisobutene epoxides by reaction with amines and subsequent dehydration and reduction of the amino alcohols, as described in particular in DE 196 20 262 A1.

Additives comprising nitro groups (ii), optionally in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 A1 and WO 96/03479 A1. These reaction products are generally mixtures of pure nitropolyisobutanes (e.g. α,β-dinitropolyisobutane) and mixed hydroxynitropolyisobutanes (e.g. α-nitro-β-hydroxypolyisobutane).

Additives comprising hydroxyl groups in combination with mono- or polyamino groups (iii) are in particular reaction products of polyisobutene epoxides obtainable from polyisobutene having preferably predominantly terminal double bonds and M_{N}=from 300 to 5000, with ammonia or mono- or polyamines, as described in particular in EP 476 485 A1.

Additives comprising carboxyl groups or their alkali metal or alkaline earth metal salts (iv) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP 307 815 A1. Such additives serve mainly to prevent valve seat wear and can, as described in WO 87/01126 A1, advantageously be used in combination with customary detergents such as poly(iso)buteneamines or polyetheramines. Additives comprising sulfonic acid groups or their alkali metal or alkaline earth metal salts (v) are preferably alkali metal or alkaline earth metal salts of an alkyl sulfosuccinate, as described in particular in EP 639 632 A1. Such additives serve mainly to pre-vent valve seat wear and can be used advantageously in combination with customary detergents such as poly(iso)buteneamines or polyetheramines.

Additives comprising polyoxy-C₂-C₄-alkylene moieties (vi) are preferably polyethers or polyetheramines which are obtainable by reaction of C₂- to C₆₀-alkanols, C₆- to C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP 310 875 A1, EP 356 725 A1, EP 700 985 A1 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (vii) are preferably esters of mono-, di- or tricarboxylic acids with long-chain alkanols or polyols, in particular those having a minimum viscosity of 2 mm²/s at 100° C., as described in particular in DE 38 38 918 A1. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Additives which comprise moieties derived from succinic anhydride and having hydroxyl and/or amino and/or amido and/or imido groups (viii) are preferably corresponding derivatives of polyisobutenylsuccinic anhydride which are obtainable by reacting conventional or highly reactive polyisobutene having M_{N}=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such gasoline fuel additives are described in particular in U.S. Pat. No. 4,849,572.

Additives comprising moieties obtained by Mannich reaction of phenolic hydroxyl groups with aldehydes and mono- or polyamines (ix) are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having M_{N}=from 300 to 5000. Such “polyisobutene-Mannich bases” are described in particular in EP 831 141 A1.

For a more precise definition of the additives detailed individually, reference is explicitly made here to the disclosures of the abovementioned prior art documents. Dispersants as component c) are, for example, imides, amides, esters and ammonium and alkali metal salts of polyisobutenesuccinic anhydrides. These compounds find use especially in lubricant oils, but sometimes also as detergents in fuel compositions. Further additives and assistants which may, if appropriate, be present as component d) of the packages are

organic solvents, for example alcohols such as methanol, ethanol, propanol, isopropanol, butanol, isobutanol, sec-butanol, pentanol, isopentanol, neopentanol or hexanol, for example glycols such as 1,2-ethylene glycol, 1,2- or 1,3-propylene glycol, 1,2-, 2,3- or 1,4-butylene glycol, di- or triethylene glycol or di- or tripropylene glycol, for example ethers such as methyl tert-butyl ether, 1,2-ethylene glycol monomethyl ether or 1,2-ethylene glycol dimethyl ether, 1,2-ethylene glycol monoethyl ether or 1,2-ethylene glycol diethyl ether, 3-methoxypropanol, 3-isopropoxypropanol, tetrahydrofuran or dioxane, for example ketones such as acetone, methyl ethyl ketone or diacetone alcohol, for example esters such as methyl acetate, ethyl acetate, propyl acetate or butyl acetate, for example lactams such as N-methylpyrrolidinone (NMP), for example aliphatic or aromatic hydrocarbons and also mixtures thereof such as pentane, hexane, heptane, octane, isooctane, petroleum ether, toluene, xylene, ethylbenzene, tetralin, decalin, dimethylnaphthalene or petroleum spirit and, for example, mineral oil such as gasoline, kerosene, diesel oil or heating oil,
corrosion inhibitors, for example based on ammonium salts, having a tendency to form films, of organic carboxylic acids or of heterocyclic aromatics in the case of nonferrous metal corrosion protection,
antioxidants or stabilizers, for example based on amines such as p-phenylenediamine, dicyclohexylamine or derivatives thereof or on phenols such as 2,4-di-tert-butylphenol or 3,5-di-tert-butyl-4-hydroxyphenylpropionic acid,
demulsifiers,
antistats,
metallocenes such as ferrocene or methylcyclopentadienylmanganese tricarbonyl,
lubricity improvers (lubricity additives) such as certain fatty acids, alkenylsuccinic esters, bis(hydroxyalkyl) fatty amines, hydroxyacetamides or castor oil,
amines for increasing the pH of the fuel,
further markers other than the compounds of the general formula I and
dyes.

The concentration of component a), i.e. of the at least one compound of the general formula (I), in the inventive packages is typically selected in such a magnitude that, after addition of the package to the mineral oil, the desired concentration of marker(s) is present therein. Typical concentrations of the markers in the mineral oil are, for instance, in the range from 0.01 up to a few 10s of ppm by weight.

Component b), i.e. the at least one carrier oil, is present in the packages typically in a concentration of from 1 to 50% by weight, in particular from 5 to 30% by weight, and component c), i.e. the at least one detergent and/or the at least one dispersant, typically in a concentration of from 25 to 90% by weight, in particular from 30 to 80% by weight, based in each case on the total amount of components a) to c) and, where present, d), the sum of the individual concentrations of components a) to c) and, if appropriate, d) adding up to 100% by weight.

When, as component d), corrosion inhibitors, antioxidants or stabilizers, demulsifiers, antistats, metallocenes, lubricity improvers and amines to reduce the pH of the fuel are present in the packages, the sum of their concentrations typically does not exceed 10% by weight, based on the total weight of the package (i.e. the total amount of components a) to c) and d)), the concentration of the corrosion inhibitors and demulsifiers being typically in the range of from in each case about 0.01 to 0.5% by weight of the total amount of the package.

When, as component d), additional organic solvents (i.e. not already introduced with the remaining components) are present in the packages, the sum of their concentrations typically does not exceed 20% by weight, based on the total amount of the package. These solvents generally stem from solutions of the markers and/or dyes, which are added to the packages instead of the pure markers and/or dyes with a view to more precise meterability.

When, as component d), further markers other than the compounds of the general formula (I) are present in the packages, their concentration is in turn based on the content that they are to have after addition of the packages in mineral oil. That which was stated for component a) applies mutatis mutandis.

When, as component d), dyes are present in the inventive packages, their concentration is typically, for instance, between 0.1 to 5% by weight, based on the total amount of the package.

The invention will be illustrated in detail by the examples without the examples restricting the subject matter of the invention.

### EXAMPLES

The compounds of the general formula (I) in the examples were prepared by the abovementioned known processes which are familiar to those skilled in the art from the literature.

Tridecylamine isomer mixture: commercial product from BASF Aktiengesellschaft.

### Example 1

### Preparation of 1,4-diamino-N-tridecyl-2,3-anthraquinonedicarboximide (Isomer Mixture)

12.8 g (0.042 mol) of 1,4-diamino-2,3-anthraquinonedicarboximide were suspended in 64 g of o-dichlorobenzene. The reaction mixture was heated to 140° C., stirred briefly (approx. 10 min) and then cooled to 60° C. At 60° C., 19.9 g (0.1 mol) of tridecylamine isomer mixture were added to the reaction mixture. The reaction mixture was then heated to 120° C. and kept at this temperature for approx. 7 h. Thereafter, the mixture was cooled again to 60° C. and admixed with 2.0 g (0.01 mol) of tridecylamine isomer mixture. Subsequently, the reaction mixture was heated again to 120° C. and kept at this temperature for approx. 3.5 h. Thereafter, another 2.0 g (0.01 mol) of tridecylamine isomer mixture were added. After a further 17.5 h at 120° C., the reaction mixture was cooled to room temperature and filtered, which left a solid, which was washed and dried in a vacuum drying cabinet. By adding approx. 200 ml of methanol, further solid was precipitated out of the filtrate, and was filtered off, washed and dried in a vacuum drying cabinet.

The combined solids were purified by column chromatography on silica gel with dichloromethane as the eluent. After the solvent had been removed, 11.3 g of blue solid were obtained.

The spectroscopic data (absorption spectrum) of the product were determined: UV/Vis: λₘₐₓ(ε)=676 nm (15400) in toluene

### Example 2

### Detection of a Marker by Detection of the Fluorescence in THF

The marker 1,4-diamino-N-isopropyl-2,3-anthraquinonedicarboximide

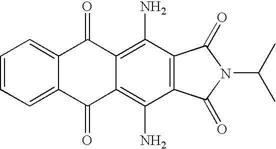

was dissolved in various concentrations in tetrahydrofuran (THF) and detected by measuring the excited fluorescence. For this purpose, the solution of the marker was excited with the aid of a laser diode which has a power of 3 mW and an excitation wavelength of 660 nm. The fluorescence was detected integrally by means of an Si photodiode at right angles to the excitation beam by a commercial edge filter with an edge wavelength of 695 nm (long pass). The following results were obtained:

| Concentration | |
|---|---|
| [ppb] | Scale divisions |
| 1000 | 2.110 |
| 500 | 1.150 |
| 200 | 0.412 |
| 100 | 0.194 |
| 50 | 0.100 |
| 20 | 0.050 |
| 10 | 0.021 |
| 0 | 0.001 |

The measured relative fluorescence intensities (scale divisions) have a very good linear correlation with the concentrations used. The square of the correlation coefficient is: 0.998.

### Example 3

### Detection of a Marker by Detection of the Fluorescence in Gasoline

The marker from Example 1 was dissolved in ARAL® Ultimate gasoline. The following results for the fluorescence intensities were obtained:

| Concentration | |
|---|---|
| [ppb] | Scale divisions |
| 1000 | 1.510 |
| 500 | 0.657 |
| 200 | 0.271 |
| 100 | 0.141 |
| 50 | 0.098 |
| 20 | 0.036 |
| 10 | 0.015 |
| 0 | 0.000 |

In this example too, the fluorescence signal (relative intensity: scale divisions) has a good linear correlation with the concentration. The square of the correlation coefficient is: 0.995.

### Example 4

### Absorption Wavelengths of Some Markers

The markers were dissolved in methylene chloride (MCL) and the longest-wavelength absorption maximum λₘₐₓ of the individual substances was determined.

| | λₘₐₓ |
|---|---|
| | (MCL) |
| Marker | [nm] |
| 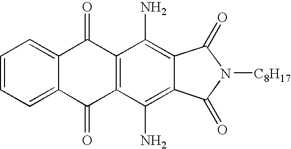 CAS: 35170-70-8 | 668 |
| 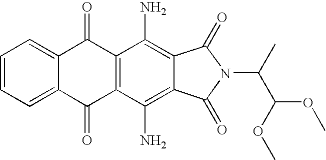 | 675 |
| 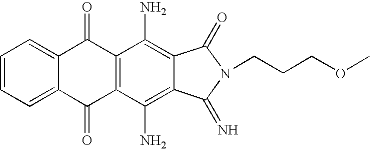 CAS: 13418-49-0 | 659 |
| 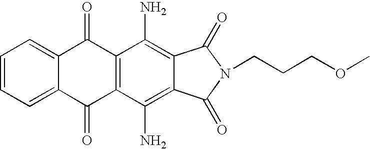 CAS: 3316-13-0 | 668 |

1: A compound of formula (I) 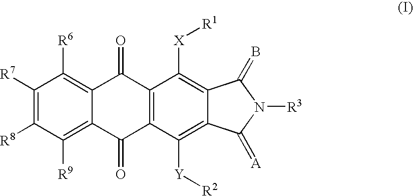
where the symbols are each defined as follows:
X, Y are each independently, identically or differently, O, NR⁴, heterocycles,
A, B are each independently, identically or differently, O, NR⁵,
M are alkali metals,
R¹, R² are each independently, identically or differently, H, C₁-C₂₀-alkyl, C₂-C₂₀-alkenyl, C₂-C₂₀-alkynyl, C₃-C₁₅-cycloalkyl, aryl, heterocycles,
R³ is C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, heterocycles,
R⁴ is H, C₁-C₂₀-alkyl, C₂-C₂₀-alkenyl, C₂-C₂₀-alkynyl, C₃-C₁₅-cycloalkyl, aryl, heterocycles,
R⁵ is H, C₁-C₂₀-alkyl, C₂-C₂₀-alkenyl, C₂-C₂₀-alkynyl, C₃-C₁₅-cycloalkyl, aryl, heterocycles, C₁-C₂₀-alkyl, C₁-C₂₀-alkylcarbonyl, C₁-C₂₀-alkoxy, C₁-C₂₀-alkoxycarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₃-C₁₅-cycloalkylcarbonyl, aryl, arylcarbonyl, aryloxy, aryloxycarbonyl, heterocycles, NR¹R², halogen, CN, NO₂,
where the substituents R¹, R², R³, R⁴, R⁵, R⁶, R⁷, R⁸ or R⁹
may each be interrupted at any position by one or more heteroatoms, where these heteroatoms number not more than 10, may be substituted in each case at any position, but not more than five times, by NR¹R², CONR¹R², COOM, COOR¹, SO₃M, SO₃R¹, CN, NO₂, C₁-C₂₀-alkyl, C₁-C₂₀-alkoxy, aryl, aryloxy, heterocycles, heteroatoms or halogen, where these may likewise be substituted not more than twice by the groups mentioned,
or may be interrupted and substituted as described above.

2: The compound according to claim 1, wherein the symbols are each defined as follows:
X, Y are each independently, identically or differently, O, NR⁴,
A, B are each independently, identically or differently, NH, O,
R¹, R² are identically or differently H, C₁-C₂₀-alkyl, aryl,
R³ is C₁-C₂₀-alkyl, C₃-C₁₅-cycloalkyl, aryl, heterocycles,
R⁴ is H, C₁-C₂₀-alkyl, aryl, heterocycles,
R⁶, R⁷, R⁸, R⁹ are each independently, identically or differently, H, C₁-C₂₀-alkyl, C₁-C₂₀-alkoxy, C₃-C₁₅-cycloalkyl, aryl, aryloxy, NR¹R², halogen, CN, NO₂.

3: The compound according to claim 1, wherein the symbols are each defined as follows:
X, Y are both identically NR⁴,
A, B are each independently, identically or differently, NH, O,
R¹, R² are each H,
R³ is C₁-C₂₀-alkyl, C₃-C₁₅-cycloalkyl, aryl,
R⁴ is H,
R⁶, R⁷, R⁸, R⁹ are each independently, identically or differently, H, C₁-C₂₀-alkyl, C₁-C₂₀-alkoxy, aryl, aryloxy, NR¹R², F, Cl, Br, CN, NO₂.

4-6. (canceled)

7: A liquid comprising at least one compound of formula (I) according to claim 1 as a marker.

8: The liquid according to claim 7, wherein the liquid is an oil.

9: The liquid according to claim 7, wherein the liquid is a mineral oil.

10: The liquid according to claim 7, wherein the liquid is an additive concentrate.

11: A method for detecting markers in liquids which comprise at least one compound of formula (I) according claim 1 in an amount which is sufficient to excite detectable fluorescence on irradiation with radiation of a suitable wavelength, comprising:
a) irradiating said liquid with electromagnetic radiation of a wavelength in the range from 500 to 900 nm; and
b) detecting the excited fluorescence radiation with a device for detecting radiation in the range from 500 to 1000 nm.

12: A method for detecting markers in liquids which comprise at least one compound of formula (I) according to claim 1 in an amount which is sufficient to exhibit detectable absorption on irradiation with radiation of a suitable wavelength, comprising:
a) irradiating said liquid with electromagnetic radiation of a wavelength in the range from 500 to 900 nm; and
b) detecting the absorption of the radiation a) with a device for detecting radiation in the range from 500 to 900 nm.

13: The method according to claim 11, wherein the liquid is an oil.

14: The method according to claim 11, wherein the liquid is a mineral oil.

15: The method according to claim 11, wherein the liquid is an additive concentrate.

16: A method for identifying liquids which comprise at least one compound of formula (I) according to claim 1 in an amount which is sufficient to excite detectable fluorescence on irradiation with radiation of a suitable wavelength, comprising:
a) irradiating said liquid with electromagnetic radiation of a wavelength in the range from 500 to 900 nm;
b) detecting the absorption of the electromagnetic radiation a) with a device for detecting radiation;
c) detecting the excited fluorescence radiation with a device for detecting radiation in the range from 500 to 1000 nm;
d) identifying said liquid with the aid of the absorption b), the fluorescence c), or a combination thereof; and
e) determining the concentration of the compound of formula (I) in the liquid with the aid of fluorescence radiation c).

17: The method according to claim 16, wherein the liquid is an oil.

18: The method according to claim 16, wherein the liquid is a mineral oil.

19: The method according to claim 16, wherein the liquid is an additive concentrate.

20: A compound of formula (I), which is a 1,4-diamino-N-tridecyl-2,3-anthraquinonedicarboximide wherein the tridecyl substituent may also be an isomer mixture of different tridecyls.

21: A compound of formula (I), which is a 1,4-diamino-N-(2,6-diisopropylphenyl)-2,3-anthraquinonedicarboximide.

22: A compound of formula (I), which is a 1,4-diamino-N-(4-dodecylphenyl)-2,3-anthraquinonedicarboximide wherein the dodecyl substituent may also be an isomer mixture of different dodecyls.

23: A compound of the formula (Ia) [chemical structure]
